(19) United States
(12) Patent Application Publication
(10) Pub. No.: US 2011/0034556 A1
(43) Pub. Date: Feb. 10, 2011

(54) USE OF TRANSGLUTAMINASE INHIBITOR IN SKIN TREATMENT

(76) Inventor: Kenneth Nicholis Dolynchuk, Winnipeg (CA)

Correspondence Address: SIM & MCBURNEY, 330 UNIVERSITY AVENUE, 6TH FLOOR, TORONTO, ON, M5G 1R7, CA

(21) Appl. No.: 12/734,824

(22) PCT Filed: Nov. 27, 2008
(86) PCT No.: PCT/CA2008/002074

§ 371 Date: Oct. 19, 2010

Related U.S. Application Data
(60) Provisional application No. 60/996,622, filed on Nov. 27, 2007.

(51) Int. Cl. A61K 8/41; A61K 8/36; A61K 8/37; A61Q 19/08; A61Q 19/00

(52) U.S. Cl. 514/552; 514/673; 514/557

(57) ABSTRACT

Human skin is treated for cosmetic purposes, in particular for the management of skin irritation and/or inflammation as well as inhibiting ageing effects, which result therefrom by using a topical cream containing at least one transglutaminase inhibitor such as putrescine.

### REFERENCE TO RELATED APPLICATION

This application claims priority pursuant to 35 USC 119(e) from U.S. Provisional Patent Application No. 60/996,622 filed Nov. 27, 2007.

### FIELD OF INVENTION

The present invention relates to skin treatment.

### BACKGROUND OF THE INVENTION

I have previously described in U.S. Pat. No. 5,885,982, a method of treating or preventing hypertrophic scar tissue in human skin by topically applying an effective amount of non-toxic amine compound which is a trans-glutaminase inhibitor having a free amine group. The amine compound that is a transglutaminase inhibitor is also selective for inhibiting Type III collagen peptide crosslinking. Treatment of hypertrophic scar generally includes the use of occlusive dressings, compression therapy, intralesional corticosteroid injections, surgical excision or cryotherapy.

### SUMMARY OF INVENTION

I have now found that transglutaminase inhibitors are useful for cosmetic purposes, specifically for the management of skin irritations and the inflammation and aging effects which result therefrom.

Accordingly, in one aspect of the present invention, there is provided a method of treatment of skin using transglutaminase inhibitors for cosmetic purposes, particularly for inhibiting aging of the skin.

### GENERAL DESCRIPTION OF INVENTION

The present invention provides a topical cream containing a trasnglutaminase inhibitor, which is used in the method of the invention. The transglutaminase inhibitor is preferably putrescine(1,4-diaminobutane), a natural metabolite, although other known transglutaminase inhibitors may be used, such as aminoacetonitrile, cadaverine (1,5-diaminopentane) and spermidine, which are all primary amines.

The topical cream is used for treating skin irritation caused by inflammation, which leads to aging of the skin. The topical cream, therefore, can be used as a cosmetic for inhibiting aging of the skin.

The provision of the transglutaminase inhibitor in the form of a topical cream provides an ease of use for the patient and is cost-effective.

One area of potential use for a composition of the invention is medicinal creams for the treatment of acne. Scarring due to skin breakouts occurs quite often and is linked to decreased self-esteem. Incorporation of a transglutaminase inhibitor into a cream to treat acne may prevent scarring from occurring.

The cream may be formulated in any convenient manner suitable for topical application. The carrier for the active component may be a mineral base, such as polyglycol, mineral oil or ointment of silicon base, preferably silicon base, and the active component may be present in the cream in an amount of about 0.05 to about 1.0% w/v of carrier, specifically about 0.8% w/v.

The use of transglutaminase inhibitors in the topical cream may be combined with other anti-aging agents, such as retinoids, for example, trans-retinoic acid and retinol palmitate (Tazoral), and Vitamin A. When such additional anti-aging agents are present, they may be used in an amount of about 0.1 to about 1% w/v for Vitamin A and about 0.05 to about 0.1 wt % for retinoids.

### EXAMPLES

A number of case studies have been conducted with human patients, as described below:

Case 1

This 68 year old female with known intrinsic allergies, presents with acute onset of blepharitis. She was seen by her family physician and was prescribed over the counter steroids and gentle cleansing.

The problem persisted for several weeks and, in fact, worsened in terms of redness and itching. The patient was treated with a topical cream in which putrescine (Sigma Chemical Co., St. Louis, Mo.) was compounded in an eutectic base (Glaxo Canola Ltd., Toronto, Ont.) at 0.8% w/v concentration (15 mM) (Fibrostat) and within 24 hours, her symptoms subsided and the erythema and irritated skin resolved completely within another few days. The patient has remained well since.

Case 2

This 80 year old rheumatoid arthritic patient sustained a sacral pressure sore and required skin grafting to close the flap donor site. Her donor site failed to heal in spite of being taken off steroids but maintained on antibiotics. She received Aquasol A (a vitamin A preparation) and later mineral oil compresses while nursed in hospital to attempt healing the wound.

After 12 weeks, it was elected to use Fibrostat in an informed trial for off label use. It was completely healed within one week and stable within 3 weeks. She was discharged shortly thereafter.

Case 3

This 48 year old female had recently undergone a chemical peel with tricloroacetic acid. The patient complained of ulceration along the mandibular border which was itchy and weepy. A trial of betamethasone was ineffective in gaining relief and, after three days, she started on Fibrostat. Immediate changes were seen in terms of resolution of irritation. Repair was clearly evident the following day and by 5 days, the redness and ulceration had completely resolved.

Case 4

This 26 year old female undergone a resurfacing peel for deep acne scars using an Erbium YAG laser. When seen the following week, she remained quite red and ulcerated, in spite of prophylaxis with antiviral medication and ELTA cream. The itchiness was another concern. Therefore, she started on Fibrostat and she resolved over the next week to go on ultimately and heal without further problem.

Case 5

The issue of the male facelift patient and visible scarring was a concern for the next patient who was a 63 year old that had undergone a facelift and simultaneous Erbium YAG resurfacing of his eyelids. The eyelids were prophylactically treated with Fibrostat and were completely healed within one week of treatment. He went on to develop hypertrophic scars in front of his ears and these were later treated with laser resurfacing and post operation Fibrostat with no evidence of visibly recurrent scars.

These patients represent the benefit of topical cream containing putrescine in helping repair irritated tissue and its role in preventing visible scar formation through the recently reported inhibition of transforming growth beta conversion. Long term effects are likely due to rapid repair seen in the epithelium as well. Whether increased apoptosis is of benefit in achieving a stable scar in short term use or not is unknown. Certainly, the quality of the scars seen clinically in prophilatic use, may also represent the effects of the creams on matrix architecture through inhibition of collagen type III crosslinking.

### SUMMARY OF DISCLOSURE

In summary of this disclosure, the present invention provides a topical cream useful for cosmetic purposes, particularly in the management of skin healing and inflammation. Modifications are provided within the scope of this invention.

1. A method for the treatment of human skin for cosmetic purposes, which comprises applying to the skin an effective amount of at least one transglutaminase inhibitor.

2. The method of claim 1 wherein said transglutaminase inhibitor is applied to the skin to inhibit aging of the skin.

3. The method of claim 1 wherein said transglutaminase inhibitor is applied to the skin for the management of skin irritation and/or the inflammation.

4. The method of claim 1 wherein said transglutaminase inhibitor is applied to the skin to the treatment of acne.

5. The method of claim 1 wherein said transglutaminase inhibitor is putrescine.

6. The method of claim 1 wherein said transglutaminase inhibitor is applied to the skin in the form of a topical cream.

7. The method of claim 6 wherein the topical cream comprises a carrier and the transglutaminase inhibitor.

8. The method of claim 7 wherein the carrier is a mineral base.

9. The method of claim 8 wherein the mineral base is a polyglycol, a mineral oil or a silicon base ointment.

10. The method of claim 7 wherein the transglutaminase inhibitor is present in the cream in an amount of about 0.05 to about 1.0% w/v of carrier.

11. The method of claim 10 wherein the transglutaminase inhibitor is present in an amount of about 0.08% w/v of the carrier.

12. The method of claim 7 wherein the topical cream further comprises at least one additional anti-aging agent.

13. The method of claim 12 wherein the at least one additional anti-aging agent is a retinoid or Vitamin A.

14. The method of claim 13 wherein the retinoid is trans-retinoic acid or retinol palmitate.

15. The method of claim 13 wherein the retinoid is present in amount of about 0.05 to about 0.1% w/v of the carrier.

16. The method of claim 13 wherein the Vitamin A is present in an amount of about 0.1 to about 1% w/v of the carrier.

17. The use of a topical cream for cosmetic treatment of skin comprising at least one transglutaminase inhibitor and a carrier for said at least one transglutaminase inhibitor.

18. The use of claim 17 wherein the carrier ins a mineral base.

19. The use of claim 18 wherein the mineral base is a polyglycol, a mineral oil or a silicon base ointment.

20. The use of claim 19 wherein said at least one transglutaminase inhibitor is present in an amount of about 0.05 to about 1.0% w/v of carrier.

21. The use of claim 20 wherein at least one the transglutaminase inhibitor comprises about 0.8% w/v of the carrier.

22. The use of claim 18 wherein the carrier further comprises at least one additional anti-aging agent.

23. The use of claim 22 wherein the at least one additional anti-aging agent is a retinoid or Vitamin A.

24. The use of claim 23 wherein the retinoid is trans-retinoic acid or retinol palmitate.

25. The use of claim 23 wherein the retinoid is present in amount of about 0.05 to about 0.1% w/v of the carrier.

26. The use of claim 23 wherein the Vitamin A is present in an amount of about 0.1 to about 1% w/v of the carrier.
